SEQUENCE LISTING (28 sequences)

SEQ ID NO: 1 - DNA, 19 bp, Artificial Sequence
  Oligonucleotide Primer
atggatgacgatatcgctg

SEQ ID NO: 2 - DNA, 19 bp, Artificial Sequence
  Oligonucleotide Primer
atgaggtagtctgtcaggt

SEQ ID NO: 3 - DNA, 20 bp, Artificial Sequence
  Oligonucleotide Primer
ggagtgtcgcttagaggtgc

SEQ ID NO: 4 - DNA, 20 bp, Artificial Sequence
  Oligonucleotide Primer
tccagaaagccaagagaagc

SEQ ID NO: 5 - DNA, 22 bp, Artificial Sequence
  Oligonucleotide Primer
tagtgaccagctataatcagag

SEQ ID NO: 6 - DNA, 20 bp, Artificial Sequence
  Oligonucleotide Primer
acgccaaggtctgaaggtcc

SEQ ID NO: 7 - DNA, 19 bp, Artificial Sequence
  Oligonucleotide Primer
ccctgctggccctgctctt

SEQ ID NO: 8 - DNA, 20 bp, Artificial Sequence
  Oligonucleotide Primer
aggtctgaaggtcacctgct

SEQ ID NO: 9 - DNA, 19 bp, Artificial Sequence
  Oligonucleotide Primer
tcatgacgtttggcaagtt

SEQ ID NO: 10 - DNA, 20 bp, Artificial Sequence
  Oligonucleotide Primer
cagaggagaaccccagatca

SEQ ID NO: 11 - DNA, 20 bp, Artificial Sequence
  Oligonucleotide Primer
gattccctatttggatcccc

SEQ ID NO: 12 - DNA, 20 bp, Artificial Sequence
  Oligonucleotide Primer
ctctctgtggcactgaacca

SEQ ID NO: 13 - DNA, 19 bp, Artificial Sequence
  Oligonucleotide Primer
ccacccagtttacaagctc

SEQ ID NO: 14 - DNA, 20 bp, Artificial Sequence
  Oligonucleotide Primer
tgtaggcagtacgggtcctc

SEQ ID NO: 15 - DNA, 20 bp, Artificial Sequence
  Oligonucleotide Primer
tgtaggcagtacgggtcctc

SEQ ID NO: 16 - DNA, 20 bp, Artificial Sequence
  Oligonucleotide Primer
ccaccccagtttacaagctc

SEQ ID NO: 17 - DNA, 20 bp, Artificial Sequence
  Oligonucleotide Primer
cattgttgcaccttgtcacc

SEQ ID NO: 18 - DNA, 20 bp, Artificial Sequence
  Oligonucleotide Primer
ttctgctgctttccctcatt

SEQ ID NO: 19 - DNA, 20 bp, Artificial Sequence
  Oligonucleotide Primer
gcaaatgtgtgtttgatgcc

SEQ ID NO: 20 - DNA, 20 bp, Artificial Sequence
  Oligonucleotide Primer
atgaccaaactcttggaccg

SEQ ID NO: 21 - DNA, 18 bp, Artificial Sequence
  Oligonucleotide Primer
cgccgcctgtccgcttcc

SEQ ID NO: 22 - DNA, 24 bp, Artificial Sequence
  Oligonucleotide Primer
ttgggcttccgttttctggtttga

SEQ ID NO: 23 - DNA, 20 bp, Artificial Sequence
  Oligonucleotide Primer
acctgagtccgagtctgacc

SEQ ID NO: 24 - DNA, 20 bp, Artificial Sequence
  Oligonucleotide Primer
ggcaccttgagaaagcagtc

SEQ ID NO: 25 - DNA, 24 bp, Artificial Sequence
  Oligonucleotide Primer
ggcgttctctttggaaaggtgttc

SEQ ID NO: 26 - DNA, 20 bp, Artificial Sequence
  Oligonucleotide Primer
ctcgaaccacatccttctct

SEQ ID NO: 27 - DNA, 23 bp, Artificial Sequence
  Oligonucleotide Primer
tgaagagagcggagaaggagatc

SEQ ID NO: 28 - DNA, 24 bp, Artificial Sequence
  Oligonucleotide Primer
tctggagttaagaaatcggagctg

(19) United States
(12) Patent Application Publication
(10) Pub. No.: US 2003/0162290 A1
(43) Pub. Date: Aug. 28, 2003

(54) Method for inducing differentiation of embryonic stem cells into functioning cells

(76) Inventors: Kazutomo Inoue, Sakyo-ku (JP); Dohoon Kim, Sakyo-ku (JP); Yanjun Gu, Sakyo-ku (JP); Michiyo Ishii, Kamigyo-ku (JP)

Correspondence Address: BIRCH STEWART KOLASCH & BIRCH, PO BOX 747, FALLS CHURCH, VA, 22040-0747, US

(21) Appl. No.: 10/054,789

(22) Filed: Jan. 25, 2002

(51) Int. Cl. C12N 5/08

(52) U.S. Cl. 435/366; 435/372

(57) ABSTRACT

The present invention provides a 4-step method for inducing differentiation of embryonic stem cells into functioning cells comprising 1) expanding ES cells; 2) inducing Embryoid Bodies in the presence of leukemia Inhibitor factor and basic FGF; 3) selection expanding of the EBs and 4) differentiation. According to the present invention, ES cells can be differentiated into either insulin producing pancreatic islet like cell clusters or nerve like cells. Thus obtained functioning cells may be potential sources of donor cells in cell transplant therapy for many patients.

### BACKGROUND OF THE INVENTION

1. Field of the Invention

The present invention relates to a method for inducing differentiation of mammalian embryonic stem cells into functioning cells. The present invention also relates to the functioning cells obtained by the present invention and a method for treatment of a patient by implanting functioning cells to the patient.

2. Art Related

Pluripotent stem cells have been derived from two embryonic sources. Embryonic stem (ES) cells are derived from the inner cell mass of preimplantation embryos, and embryonic germ (EG) cells are derived from primordial germ cells (PGCs). Both ES and EG cells are pluripotent and demonstrate germ-line transmission in experimentally produced chimeras. Mouse ES and EG cells share several morphological characteristics such as high levels of intracellular alkaline phosphatase (AP), and presentation of specific cell surface glycolipids and glycoproteins. These properties are characteristic of, but not specific for, pluripotent stem cells. Other important characteristics include growth as multicellular colonies, normal and stable karyotypes, the ability to be continuously passaged, and the capability to differentiate into cells derived from all three embryonic germ layers. Pluripotent stem cell lines that share most of these characteristics also have been reported for chicken, mink, hamster, pig, rhesus monkey, and common marmoset. Also a stem cell is a cell that has the ability to divide (self-replication) for indefinite periods—often throughout the life of the organism. Under the right conditions, or given the right signals, stem cells can give rise (differentiate) to the many different cell types that make up the organism.

Recently, S. H. Lee et al. (Nature Biotechnology 18, 675-679 (2000), the disclosure of the publication is herein incorporated by reference) disclosed to generate CNS progenitor populations from ES cells, to expand these cells and to promote their differentiation into dopaminergic and serotonergic neurons in the presence of mitogens and specific signaling molecules. The differentiation and maturation of neuronal cells was completed after mitogen withdrawal from the growth medium. This experimental system provides a powerful tool for analyzing the molecular mechanisms controlling the functions of these neurons in vitro and in vivo, and potentially for understanding and treating neurodegenerative and psychiatric diseases.

Also, H. Kawasaki et al. (Neuron 28, 31-40(2000), the disclosure of the publication is herein incorporated by reference) have identified a stromal cell-derived inducing activity (SDIA) that promotes neural differentiation of mouse ES cells. SDIA accumulates on the surface of PA6 stromal cells and induces efficient neuronal differentiation of co-cultured ES cells in serum-free conditions without use of either retinoic acid or embryonic bodies. BMP4, which acts as an antineuralizing morphogen in Xenopus, suppresses SDIA-induced neuralization and promotes epidermal differentiation. A high proportion of tyrosine hydroxylase-positive neurons producing dopamine are obtained from SDIA-treated ES cells. When transplanted, SDIA-induced dopaminergic neurons integrate into the mouse striatum and remain positive for tyrosine hydroxylase expression. Neural induction by SDIA provides a new powerful tool for both basic neuroscience research and therapeutic applications.

In a study of B. Soria et al., mouse embryonic stem cells have been introduced as a new potential source for cell therapy in type I diabetic patients (Diabetes 49: 157-162 (2000), the disclosure of the publication is herein incorporated by reference). Using a cell-trapping system, they have obtained an insulin-secreting cell clone from undifferentiated ES cells. The construction used allows the expression of a neomycin selection system under the control of the regulatory regions of the human insulin gene. The chimeric gene also contained a hygromycin resistance gene (pGK-hygro) to select transfected cells. A resulting clone (IB/3x-99) containing 16.5 ng/μg protein of total insulin displays regulated hormone secretion in vitro in the presence of various secretagogues. Clusters obtained from this clone were implanted in the spleen of streptozotocin-induced diabetic animals. Hyperglycemia of the transplanted animals were normalized within one week and their body weight were restored in 4 weeks. Whereas slower recovery was observed in the transplanted animals than control mice in an intraperitoneal glucose tolerance test, blood glucose levers after meal load were normalized in a similar manner. This approach opens new possibilities for tissue transplantation in the treatment of typel and type 2 diabetes and offers an alternative to gene therapy.

S. Assady et al. (Diabetes 50: 1691-1697 (2001), the disclosure of the publication is herein incorporated by reference), used pluripotent undifferentiated human embryonic stem cells (hES) as a model system for lineage-specific differentiation. They cultured hES cells in both adherent and suspension culture conditions, and observed spontaneous in vitro differentiation of the cells including generation of cells with characteristics of insulin-producing β-cells. Immunohistochemical staining for insulin was observed in a surprisingly high percentage of the cells. Secretion of insulin into the medium was observed in a differentiation-dependent manner and was associated with the appearance of other β-cell markers. These findings suggest that the hES cell model system is a potential basis for enrichment of human β-cells or their precursors, as a possible future source for cell replacement therapy in diabetes.

Su-Chun Zhang et al. (Nature Biotech. 19, 1129-1133 (2001), the disclosure of the publication is herein incorporated by reference) disclose in vitro differentiation, enrichment, and transplantation of neural precursor cells from human ES cells. Upon aggregation to embryoid bodies, differentiating ES cells formed large numbers of neural tube-like structures in the presence of fibroblast growth factor 2 (FGF-2). Neural precursors within these formations were isolated by selective enzymatic digestion and further purified on the basis of differential adhesion. Following withdrawal of FGF-2, they differentiated into neurons, astrocytes, and oligodendrocytes. After transplantation into the neonatal mouse brain, human ES cell-derived neural precursors were incorporated into a variety of brain regions, where they differentiated into both neurons and astrocytes. No teratoma formation was observed in the transplant recipients. These results depict human ES cells as a source of transplantable neural precursors for possible nervous system repair.

Nadya Lumelsky et al. disclose a series of experiments in which they induced mouse embryonic cells to differentiate into insulin-secreting structures that resembled pancreatic islet (Science 292, 1389-1394 (2001), the disclosure of the publication is incorporated herein by reference). They have generated cells expressing insulin and other pancreatic endocrine hormones from mouse ES cells. The cells self-assemble to form three-dimensional cluster similar in topology to normal pancreatic islets where pancreatic cell types are in close association with neurons. Glucose triggers insulin release from these cell clusters by mechanisms similar to those employed in vivo. When injected into diabetic mice, the insulin-producing cells undergo rapid vascularization and maintain a clustered, islet-like organization.

However, the insulin-producing cells obtained by Lumelsky did not express pancreatic specific markers, amylase and carboxypeptidase. Further, Lumelsky grafted the insulin-producing cells into a diabetic model animal but failed to observe a sustained correction of hyperglycemia in the model animal.

Seven million people in Japan and 16 million people in the United States are affected by type I diabetics. At present, daily insulin administration or allogenic pancreas transplantation is employed for treatment of diabetics. Although the overall success rates of the pancreas transplantation have significantly increased, organ transplantation requires very invasive surgery and life-long immunosurpressive treatments, which significantly strain the patient. Further, availability of donor organs is still serious problem preventing the operation to be popular. Therefore, development of a simple and universal treatment for diabetes is desired.

### SUMMARY OF THE INVENTION

One object of the present invention is to provide a novel method for inducing differentiation of pluripotent embryonic stem cells into functioning cells, especially pancreatic islet like cell clusters and nerve like cells.

Another object of the present invention to provide a method for treating a patient having disorders in pancreatic islet function.

Another object of the present invention is to provide a method for treating a patient having neuronal degeneration or spinal code disorders.

Further object of the present invention is to provide functioning cells which are derived from mammalian ES cells and exhibit pancreatic islet like or nerve like functions.

Accordingly, the present invention provides a method for inducing differentiation of mammalian embryonic stem cells into functioning cells, which comprises the steps of;

1) culturing the mammalian embryonic stem cells together with feeder cells with a medium comprising leukemia inhibitor factor;

2) culturing the obtained cells in absence of feeder cells with a medium comprising leukemia Inhibitor factor and basic fibroblast growth factor (hereinafter referred to as “bFGF”) in suspension culture condition to give embryonic bodies;

3) culturing the obtained embryonic bodies with a growth-selection medium; and

4) culturing the obtained cell clusters with a differentiation medium to give functioning cells.

According to the present invention, functioning cells such as pancreatic islet like cell clusters and nerve like cells can be differentiated from the mammalian ES cells.

The pancreatic islet like cell clusters induced by the present invention have an ability to produce insulin and to secrete insulin in response to glucose stimulation, and the cells consisting the clusters express pancreatic-related endocrine and exocrine markers including insulin, glucagon, Glut-2, islet amyloid polypeptide, amylase and carboxypeptitase.

The nerve like cells induced by the present invention exhibit nerve fiber like appearance and the cells consisting the clusters express nerve related markers including nestin, β-tublin III, seletonin, tyrosin hydroxylase Nurt 1.

The inventors grafted the insulin-secreting islet like cell clusters induced from mouse ES cells by the method of the present invention into streptozotocine induced diabetic mice, and succeeded in decreasing the high blood glucose levels of the diabetic mice to those around the normal level. This study supports that the insulin producing islet like cell clusters obtained by the invention are useful for treatment of diabetics.

The present invention further provides a method for treating a mammalian patient having disorders in pancreatic islet function, which comprises the step of transplanting islet-like cell clusters induced from allogenic ES cells according to the invention to the patient.

The present invention also provides a method for treating a patient with nerve degenerative disease or spinal cord injury, which comprises the step of transplanting nerve like cells induced from allogenic ES cells according to the present invention to the patient.

Further, the present invention also provides functioning cells including pancreatic islet like cell clusters and nerve like cells derived from the mammalian ES cells by the method of the present invention. The functioning cells are useful not only for cell transplant therapy but also for in vitro screening of various new drugs which affect or restore islet or nerve function, safety evaluation of new drugs and so on.

### BRIEF DESCRIPTION OF THE DRAWINGS

FIG. 1 is a schematic description of differentiation steps of the present invention from ES cells to functioning cells.

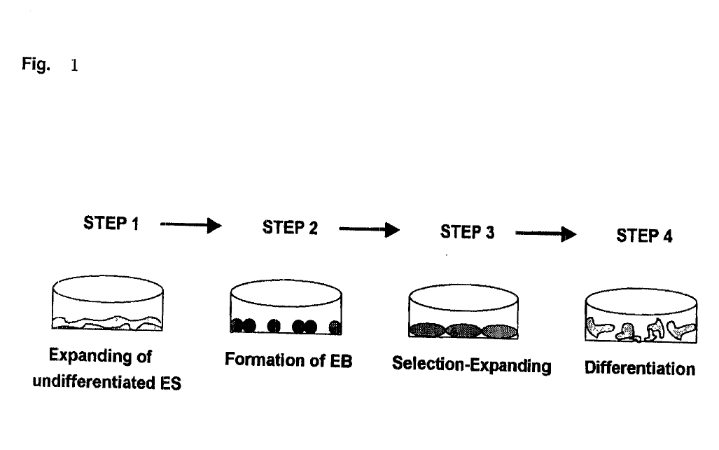

FIG. 2 represents result of insulin secretion from the cell clusters obtained in Example 1 in response to glucose stimulation. In this graph, column L represents the amount of insulin secreted per cluster in response to low dose (3.3 mg/L) glucose stimulation determined 5 minutes and 30 minutes respectively after the stimulation. Column H represents the amount in response to high dose (25 mmol/L) glucose stimulation.

FIG. 3 represents time-course of non-fasting blood glucose levels of diabetic mice implanted with the pancreatic islet like cell clusters derived from mouse ES cells compared to that of sham operation group.

FIG. 4 represents time course of body weight of diabetic mice implanted with the pancreatic islet like cell clusters derived from ES cells compared to that of sham operation group.

### DETAILED DESCRIPTION OF THE INVENTION

In the specification, claims and drawings of the instant application, the term “embryonic stem cell(s)” or “ES cell(s)” represents pluripotent cells derived from the inner cell mass of in vitro fertilized blastocytes.

Embryoid body or EB represents a cell cluster composed of three embryonic germ layers and formed from ES cells on their in vitro aggregation.

The feeder cell layer as used herein is constructed in accordance with procedures known in the art, and may be prepared from mice fatal fibroblast cells. Feeder cells are now, commercially available.

The mammalian ES cells which may be used herein are not limited and may be rodent, such as mouse ES cells and rat ES cells, as well as primate such as cynomolgus ES cells and human ES cells. At present, various ES cells are derived and available including mice and human EC cells. Alternatively, the ES cells used herein may be those obtained from mammalian fertilized ovum by means of previous reports. For example, techniques for isolating stable cultures of human embryonic stem cells have been described by Thomson et al. (U.S. Pat. Nos. 5,843,780 and 6,200,806; Science vol. 282 1145-1147 (1998), the disclosure of these publications are herein incorporated by reference).

Step 1 of the present method is a conventional ES cell propagation step, which is described in, such as, N. Lumelsky et al., Science 292, 1389-1394 (2001), the disclosure of the publication is herein incorporated by reference.

Typically, mouse fetal feeder cells are cultured on a gelatin coated cell culture container to give a layer on the inner surface, then the ES cells are plated on the layer and cultured with an ES cell proliferating medium comprising leukemia inhibiting factor (hereinafter, referred to as “LIF”). By culturing under such condition as above, ES cells proliferate in an undifferentiated state.

In the method of the present invention, feeder cells may be those commercially available cells or those derived from mice fetal fibroblast cells by a conventional manner.

The ES cell proliferating medium used in step 1 may comprise 100-10000 U/ml of LIF. As a medium used in this step, any known medium that contains LIF and is useful for ES cell proliferation can be employed. An especially preferable medium is high glucose Dulbecco's modified Eagle's medium (Life Technology (herein below, Life Tech,), Grand, N.Y.) supplemented with 20% fetal bovine serum replacement (Life Tech.), 2% nonessential amino acid (Life Tech.), 0.1 mmol/l 2-mercaptoethanol (Life Tech.), 1000 U/ml of leukemia inhibitor factor (LIF; Life Tech.) and 2 mmol/1 L-glutamine (Life Tech.).

In step 1, culture of the ES cells may be continued until a desired amount of the cells is obtained. Typically, 3-7 days culture may provide enough cells. The obtained ES cells are transferred to the next step.

Throughout the inducing method of the present invention, culture of the cells or cell clusters may be carried out under a conventional cell culture condition such as at 37° C., in a humidified atmosphere of 5% CO₂ in 95% air.

In step 2, the proliferated ES cells are kept in suspension culture with a medium supplemented with LIF and bFGF. Heretofore, LIF has been believed to help retain the ES cells in an undifferentiated state and the art has believed that it is indispensable to exclude LIF from the culture in order to induce differentiation of the ES cells. Accordingly, as far as known to the inventors, all of the proposed EB inducing conditions contain the step culturing the expanded ES cells in suspension culture with a medium containing no LIF to allow their aggregation (for example, Su-Chen Zhang et al., Nature biotechnology, 19, 1129-1133 (2001), the disclosure of the publication is herein incorporated by reference).

The present inventors, however, succeeded to provide highly efficient EB formation from the ES cells with a medium comprising LIF and bFGF.

The medium used in step 2 contains LIF and bFGF. The amount of LIF in the medium may preferably be about 100-10000 U/ml. The amount of bFGF in the medium may preferably be about 2-100 ng/ml. The medium used in this step may further comprise one or more growth factors such as activin and nerve growth factor, cytokines such as interleukin-1 and interleukin-2, vitamins such as rethinol and nicotinamide, additional amino-acids such as tyrosine and lysine, and extra cellular matrixes such as fibronectin, aminin, collagen and heparin in a conventional chemically defined cell culture medium. An example of especially preferred medium used in this step is high glucose Dulbecco's modified Eagle's medium (Life Tech.) supplemented with 20% fetal bovine serum replacement (Life Tech.), 2% nonessential amino acid (Life Tech.), 0.1 mmol/l 2-mercaptoethanol (Life Tech.), 1000 U/ml of leukemia inhibitor factor (LIF; Life Tech.), 2 mmol/l of L-glutamine (Life Tech.) and 4 ng/ml of bFGF (R&D systems, Minneapolis).

In step 2, the ES cells are cultured in suspension without the feeder cell layer to allow the cells aggregate to give embryoid bodies. The formation of EBs may be microscopically monitored. According to the present invention, EB formation may be observed from 2 days of the suspension culture. The suspension culture may be continued for 5-10 days to obtain enough amount of EBs. According to the present invention, a significantly larger number of vital EBs are induced than those induced by a conventional suspension culture step with a medium containing no LIF nor bFGF.

The EBs obtained in step 2 are then transferred to a selection-expansion step (step 3). In step 3, thus obtained EBs are plated on a culture container of which inner surface is coated with a protein, such as collagen type IV, and cultured with an appropriate selection-expansion medium. It is preferable to culture the EBs in the protein coated container with the medium used in step 2 for about 2 days and then exchange the medium with a selection-expanding medium.

The selection-expanding medium used in step 3 may preferably be a serum-free cell culture medium supplemented with nicotinamide, insulin and fibronectine. The medium used in this step may further comprise one or more growth factors such as activin and nerve growth factor, cytokines such as interleukin-1 and interleukin-2, vitamins such as rethinol, additional amino-acids such as tyrosine and lysine, and extra cellular matrixes such as laminin, collagen and heparin in a conventional chemically defined cell culture medium. An example of preferable medium is a serum free DMEM/F-12 medium supplemented with nicotinamide, fibronectine, and N-2 supplements (GIBCO, 17502-014: consisting of Insulin 500 μg/ml, Human transferin 10000 μg/ml, Progesterone 0.63 μg/ml, Putrescine 1611 μg/ml and Selenite 0.52 μg/ml in water).

In step 3, the EBs may be cultured with the selection-expanding medium for 3-14 days, preferably for 4-7 days.

The cell clusters obtained in step 3 are then dissociated from the container and plated on a culture container of which inner surface is coated with a protein or an amino acid. The transferred clusters are further cultured in a differentiation medium.

In step 4, the cell clusters can be differentiated into either pancreatic islet like cell clusters or nerve like cells.

In case the islet like cell clusters are desired, the cell clusters may be cultured with a serum-free cell culture medium supplemented with nicotinamide, insulin and laminine. The medium may further comprise one or more growth factors such as activin and nerve growth factor, cytokines such as interleukin-1 and interleukin-2, vitamins such as rethinol, additional amino-acids such as tyrosine and lysine, and extra cellular matrixes such as fibronectin, collagen and heparin in a conventional chemically defined cell culture medium. An especially preferred example is serum-free DMEM/F12 medium supplemented with nicotinamide, laminine and N-2 supplement.

According to the present invention, in order to differentiate into nerve like cells, the cell clusters may be cultured with a serum-free cell culture medium supplemented with lysine and laminine. The medium may further comprise one or more growth factors such as activin and nerve growth factor, cytokines such as interleukin-1 and interleukin-2, vitamins such as rethinol and nicotinamide, additional amino-acids such as tyrosine and lysine, and extra cellular matrixes such as fibronectin, collagen and heparin in a conventional chemically defined cell culture medium. An especially preferred example is serum-free DMEM/F12 medium supplemented with lysine, laminine and N-2 supplement.

In step 4, cell clusters may be cultured for 3-90 days or longer. 4-12 days culture will be enough for differentiation into the desired functioning cells and further culture may provide further proliferation of the differentiated clusters.

The pancreatic islet like cell clusters obtained by the present invention represent an ability to produce insulin and to secret insulin in response to glucose stimulation, and the cells consisting the clusters express genes specific to pancreatic endocrine cells including insulin, glucagon, Glut-2 and islet amyloid polypeptide as well as those specific to pancreatic exocrine cells including amylase and carboxypeptitase.

The nerve like cells obtained by the present invention represent nerve fiber like appearances and express markers relevant to nerve cells including nestin, b-tublin III, seletonin, tyrosine hydroxylase. Therefore, said nerve like cells are capable of generating mature neurons.

Since mice ES cells as well as human ES cells proliferate in vitro in an undifferentiated state retaining the pluripotency for more than one year, the present method can be employed to provide enough amount of donor cells used in the cell transplanting therapy.

The present invention further provide a method for treating a mammalian patient having disorders in pancreatic islet function, which comprises transplanting islet-like cell clusters induced from allogenic ES cells according to the invention to the patient In the present invention, “mammalian patient having disorders in pancreatic islet function” includes, but not limited to, type I diabetic patient, pancreatomized patient and insulin-required diabetic patient such as type II diabetic patient or patient with cystic fibrosis. The mammalian patient may include human patient.

In this embodiment, transplantation of the pancreatic islet like cell clusters obtained as above may be carried out according to a clinically performed or proposed islet transplantation protocol (for example, Kazutomo Inoue and Masaaki Miyamoto, J. Hepatobiliary Pancreat. Surg. 7: 163-177 (2000), and Wenjing Wang et al., Transplantation 73: 122-129 (2002); the disclosure of the publications are herein incorporated by reference). For example, the pancreatic islet like cell clusters may be implanted intraportally into the liver. Alternatively, the pancreatic islet like cell clusters may be implanted into a prevascularized subcutaneous site. Said clusters may be macroencapsulated with a bio-compatible material before implantation. The amount of the clusters to be transplanted will be determined by the art based on the titer of the obtained clusters as well as the general conditions, age, sex, body weight of the patient to be treated.

Further more, the present invention also provides a method for treating a mammalian patient having disorders in nerve function, which comprises a step of transplanting the nerve like cells derived from allogenic ES cells to the patient. In the present invention, “mammalian patient having disorders in nerve function” includes, but not limited to, patients having nerve degeneration disease such as Alzheimer's disease and Creutzfeldt-Jakob disease or spinal injury. The mammalian patient may include human patient.

The present invention will be further illustrated by the following Examples. The examples are intended to illustrate but not in any means to limit the invention.

### EXAMPLE 1

Differentiation of Pancreatic Islet Like Clusters from Mouse ES Cells

Step 1

Expanding of Undifferentiated ES Cells

In this example, mouse ES cell line 129sv (passages 11; Dainippon Pharmaceutical Co. Ltd., Osaka Japan) was used. A similar study was carried out with mouse ES cell clonal line derived from C57/BL6 mouse (passage 11; kindly provided by Professor Norio NAKATSUJI of Institute for frontier medical sciences, Kyoto University, Kyoto Japan) and similar results as below were obtained (data not shown).

Mammalian ES cells can be proliferated in an undifferentiated state if they are cultured on a feeder layer in the presence of leukemia inhibitor factor. Mouse embryo feeder cells (Dainippon Pharmaceutical Co. Ltd., Osaka, Japan) which had been mitotically inactivated with 20 μg/ml mitomycin were used. ES cell culture medium of high glucose Dulbecco's modified Eagle's medium (D-MEM Cat# 12100: Life Technology, Grand, N.Y.) supplemented with 20% fetal bovine serum replacement (Life Tech.), 2% nonessential amino acid (Life Tech.), 0.1 mmol/l 2-mercaptoethanol (Life Tech.), 1000 U/ml of leukemia inhibitor factor (LIF; Life Tech.) and 2 mmol/l L-glutamine (Life Tech.) was used.

A feeder layer of the mitomycin treated mouse embryonic fibroblasts was prepared on a gelatin-coated culture dish (6 cm), 5 ml of the medium was added thereto and 10⁶ of ES cells were plated on the layer. Cells were cultured at 37° C. in humidified atmosphere of 5% CO₂ in 95% air. Every 3 days, the cells were removed from the dish by means of 0.05% trypsin solution in 0.04% EDTA (Life Tech.) and passaged into a freshly prepared medium on a freshly prepared feeder layer. The ES cells were cultured for 3-7 days.

Step 2

Formation of Embryoid Bodies (EBs)

The ES cells were disassociated by means of the trypsin-EDTA solution and were plated on a non-adherent culture dish to give cell density of 6×10⁵ cells/cm². The cells were kept in suspension culture in the medium used in the above step 1 in the absence or presence of bFGF (4 ng/ml; R&D Systems, Minneapolis, U.S.A. and Kaken Pharmaceuticals, Co. Ltd., Tokyo Japan). Cells were cultured at 37° C. in humidified atmosphere of 5% CO₂ in 95% air. Every 2 days, the media were replaced with freshly prepared ones.

The cultures were daily observed microscopically. At day 2, the cells cultured with bFGF started to aggregate to generate EBs. The suspension culture was kept for 5 days. At day 5, significantly larger number of cell clusters, i.e. EBs were observed in the culture with bFGF than those previously obtained by the conventional EB inducing process without LIF and bFGF(data not shown). In the group without bFGF, only a few aggregation was observed.

Step 3

Selection—Expanding of EBs

The EBs obtained in step 2 with the bFGF containing medium were plated on a Type IV collagen (Sigma, St. Louis, Mo.) coated 6 cm dish filled with the medium used in step 2. After cultured for 48 hr, the medium was replaced with selection-expanding medium of serum free DMEM/F-12(1:1) medium (cat#11320, Life Tech.) supplemented with 500 μg/ ml of Bovine Insulin, 1 μg/ml of Progestron, 1600 μg/ml of Putrescine and 5 μg/ml of Fibronectin and 10 mM of nicotinamide. The cell clusters were cultured for more than 7 days. During the culture, the medium was replaced with freshly prepared one every 3 days.

Step 4

Differentiation of the Cells

After 7 days culture with the selection- expanding medium, further differentiation was induced by culturing the cell clusters with serum free DMEM/F-12(1:1) medium (cat#11320, Life Tech.) supplemmented with 500 μg/ml of Bovine Insulin, 1 μg/ ml of Progestron, 1600 μg/ml of Putrescine, 1 μg 1 ml of Laminin and 10 mM of nicotinamide. The cells were incubated at 37° C. in humidified atmosphere of 5% CO₂ in 95% air for 12 days to give islet like cell clusters of about 100-400 μm in diameter.

RNA Extraction and RT-PCR Analysis

At the end of every step as above, pancreatic relating gene expression on the cells was examined by means of RT-PCR analysis.

Cellular RNA of the cells obtained in each step was isolated using ISOGEN (Nippon Gene; Osaka, Japan) according to the manufacturer's instruction. The cells were homogenized in 0.8 ml of ISOGEN using a Potter homogenizer at 4° C. The homogenate was mixed with 1 ml of chloroform, and RNA in the aqueous phase was precipitated with the same volume of isopropyl alcohol. Synthesize of cDNA was carried out with oligo dT primers (Takara Shuzo Co. Ltd., Kyoto, Japan) and Moloney murine leukemia virus (M-MLV) Superscript II reverse transcriptase (Gibco/BRL) following the manufacturer's instructions.

Based on thus obtained cDNAs, expression levels of transcription factor mRNAs were determined by means of PCR method. PCR was carried out using standard protocols with Taq polymerase (Boehringer-Mannheim, Indianapolis, Ind.). Cycling parameters were as follows, denaturation at 94° C. for 1 min, annealing at 52-61° C. for 30-120 seconds (depending on the primer) for 1 min, and elongation at 72° C. for 1 min. The number of cycles varied between 25 and 40, depending on the particular mRNA abundance. The number of cycles and the amount of cDNA were chosen in such a way as to select PCR conditions on the linear portion of the reaction curve avoiding “saturation effects” of PCR. Obtained PCR products were confirmed by sequencing.

Primer sequences (forward and reverse), and the length of the amplified products were as follows:

β-actin:
ATGGATGACGATATCGCTG
ATGAGGTAGTCTGTCAGGT
569 bp
nestin:
GGAGTGTCGCTTAGAGGTGC
TCCAGAAAGCCAAGAGAAGC
327 bp
insulin-I:
TAGTGACCAGCTATAATCAGAG
ACGCCAAGGTCTGAAGGTCC
288 bp
insulin-II:
CCCTGCTGGCCCTGCTCTT
AGGTCTGAAGGTCACCTGCT
212 bp
glucagon:
TCATGACGTTTGGCAAGTT
CAGAGGAGAACCCCAGATCA
202 bp
Islet Amyloid Polypeptide (IAPP):
GATTCCCTATTTGGATCCCC
CTCTCTGTGGCACTGAACCA
221 bp
Glucose transporter 2 (Glut2):
CCACCCAGTTTACAAGCTC
TGTAGGCAGTACGGGTCCTC
325 bp
PDX-1:
TGTAGGCAGTACGGGTCCTC
CCACCCCAGTTTACAAGCTC
325 bp
amylase-2A
CATTGTTGCACCTTGTCACC
TTCTGCTGCTTTCCCTCATT
300 bp
carboxypeptidase A:
GCAAATGTGTGTTTGATGCC
ATGACCAAACTCTTGGACCG
521 bp
GATA-4:
CGCCGCCTGTCCGCTTCC
TTGGGCTTCCGTTTTCTGGTTTGA
193 bp
HNF3:
ACCTGAGTCCGAGTCTGACC
GGCACCTTGAGAAAGCAGTC
345 bp
OCT-4:
GGCGTTCTCTTTGGAAAGGTGTTC
CTCGAACCACATCCTTCTCT
293 bp

Results are shown in table 1 below;

TABLE 1
Gene expression on the cells cultured in the presence of
bFGF in step 2

| | Step 1 | Step 2 | Step 3 | Step 4 |
|---|---|---|---|---|
| OCT4 | + | − | − | − |
| HNF-3β | + | + | + | + |
| Nestin | ± | + | ± | ± |
| Insulin-I | + | + | + | + |
| Insulin-II | + | + | + | + |
| IAPP | − | ± | ± | + |
| GATA4 | + | + | + | + |
| PDX-1 | + | ± | ± | + |
| Amylase | − | − | − | + |
| Carboxypeptiase | − | − | − | + |
| Glut 2 | + | + | + | + |
| Glucagon | − | − | − | + |

The gene of pancreatic transcription factor PDX-1, which is indispensable for pancreatic development, was expressed in steps 1 and 4 cells. Oct-4, which relates to differentiation of ES cell, was expressed in step 1 cells and down-regulated with the differentiation of the ES cells. The ES cells at every step expressed a maker of definitive (embryonic) and visceral (extra-embryonic) endoderm GATA-4 and definitive endoderm HNF3β concerning markers of pancreatic βcell fate. Nestin, a transcription factor relates to immature hormone-negative pancreatic cells, was strongly expressed in step 2 and down-regulated with the differentiation of the ES cells. The results showed that many nestin positive progenitor cells were contained in the EBs obtained in the presence of bFGF. Interestingly, EBs induced by bFGF treatment expressed transcription factors of endocrine (Insulin I, Insulin II, Glucagon, Glucose transporter-2 (Glut-2) and Islet Amyloid Polypeptide) specific genes whereas any gene concerning pancreatic islet cells did not expressed in the cell clusters obtained in step 2 using the medium without bFGF. In steps 3 and 4, the cells expressed exocrine specific genes (amylase and carboxypeptidase). These results indicates that pancreatic islet like cell clusters of the invention can be matured to a pancreatic tissue structure, which composed of endocrine cells including glucagon-producing α cells, insulin-producing β cells, pancreatic polypeptide-producing γ cells, and somatostin-producing δ cells and exocrine cells.

Insulin Secretion Test

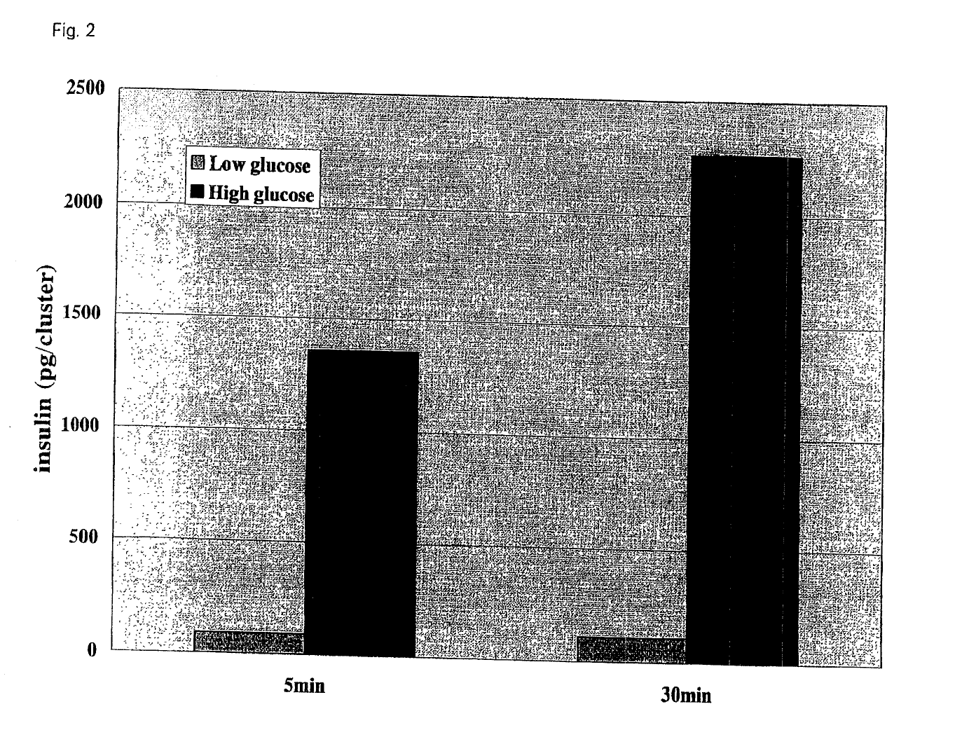

The cell clusters obtained in step 4 (20-25 clusters) were washed 3 times with PBS(−) and plated on a 6 cm cell culture dish containing Krebs-Ringer with bicarbonate buffer consisting of 120 mM NaCl, 5 mM KCl, 2.5 mM CaCl₂, 1.1 mM MgCl₂, 25 mM NaHCO₃ and 0.1% bovine serum albumin, and incubated at 37° C. 3.3 mmol/l(L) or 25 mmol/l (H) glucose was added thereto and incubated. Five and thirty minutes after the glucose stimulation, the insulin contents in the buffer were measured using insulin enzyme-linked immunosorbence assay (ELISA) kit (ALPCO, Windham, N.H.). Results are shown in FIG. 2. In the FIG. 2, the amounts of insulin secreted per one cluster in response to the low or high glucose stimulation at 5 and 30 minutes after the stimulation are shown. The clusters exhibited insulin secretion in response to glucose stimulation in a dose dependent manner.

For determination of total cellular insulin content, the cell clusters obtained in step 4 were extracted with acid ethanol (10% glacial acetic acid in absolute ethanol) overnight at 4° C., followed by cell sonication and then, the insulin content in the supernatant was determined by means of the ELIZA kit. Total cellular protein amount was determined using DC protein assay system (Bio-Rad laboratories, Hercules, Calif.). The total cellular insulin content of those cell clusters was 71.3 ng/mg protein.

Histological and Immunohistochemistry Analysis

Paraffin slices of the cell clusters obtained in step 4 were prepared as follows. The cell clusters (in step 4, incubated 12 days) were washed three times with ice-cold PBS and were fixed with methanol/aceton (1:1) for over night. The clusters were dehydrated with aqueous alcohol (70-100%), then embedded in a paraffin block and the block was sliced to give 4 μm and 8 μm thick slices.

Thus obtained 4 μm thick slices were histologically evaluated with hematoxylinleosin staining.

In order to immunohistochemical evaluation, 8 μm thick slices were stained with antibodies by means of the standard protocol. Primary antibodies used herein were follows:

nestin rabbit polyclonal 1:500 (Dako, Carpinteria, Calif.), tubulin type III (TuJ1) mouse monoclonal 1:500 (Babco, Richmond, Calif.), tubulin type III (TuJ1) rabbit polyclonal 1:2000 (Babco, Richmond, Calif.), insulin mouse monoclonal 1:1000 (Sigma, St. Louis, Mo.), insulin guinea pig polyclonal 1:100 (DAKO, Carpinteria, Calif.), glucagon rabbit polyclonal (DAKO, Carpinteria, Calif.).

In order to detect the primary antibodies, fluorescently labeled secondary antibodies (Jackson Immunoresearch Laboratories, West Grove, Pa.) were used according to the supplier's instruction.

The obtained insulin producing cell cluster was strongly positive to insulin and glucagone, and positive to nestin and TuJ1.

### EXAMPLE 2

Transplantation of the Insulin Producing Cell Clusters Into STZ Derived Diabetic Mice

The insulin producing pancreatic islet like cell clusters were transplanted to determine if the cluster could differentiate into functioning pancreatic islet in vivo.

All animal studies were carried out in accordance with Guideline for Animal Experiments of Kyoto University. Experimental diabetic mice were prepared according to the method disclosed in H. Iwata et al., Pancreas vol. 23(4) 375-381(2001), the disclosure of the publication is herein incorporated by reference. Streptozotocin (STZ) cryopreserved powder (Sigma, St. Louis, Mo.) was dissolved in 0.1 M citrate buffer, pH 4.5 before use. The STZ solution was intraperitoneally injected (227 mg/kg of body weight) to 8- to 10-weeks-old male Nude mice (Shimizu, Kyoto, Japan), Stable hyperglycemia, i.e. increased blood glucose levels of about 350-600 mg/dl) were usually developed 7 to 10 days after the STZ single injection.

Blood glucose level of the mouse was determined using Glucometer Elite XL blood glucose meter (Fujii Corp., Tokyo, Japan). Animals represent 350 mg/dl or more non-fasting blood glucose at 7-10 days of STZ injection were regarded as diabetic mice and used at 14 day from the STZ injection.

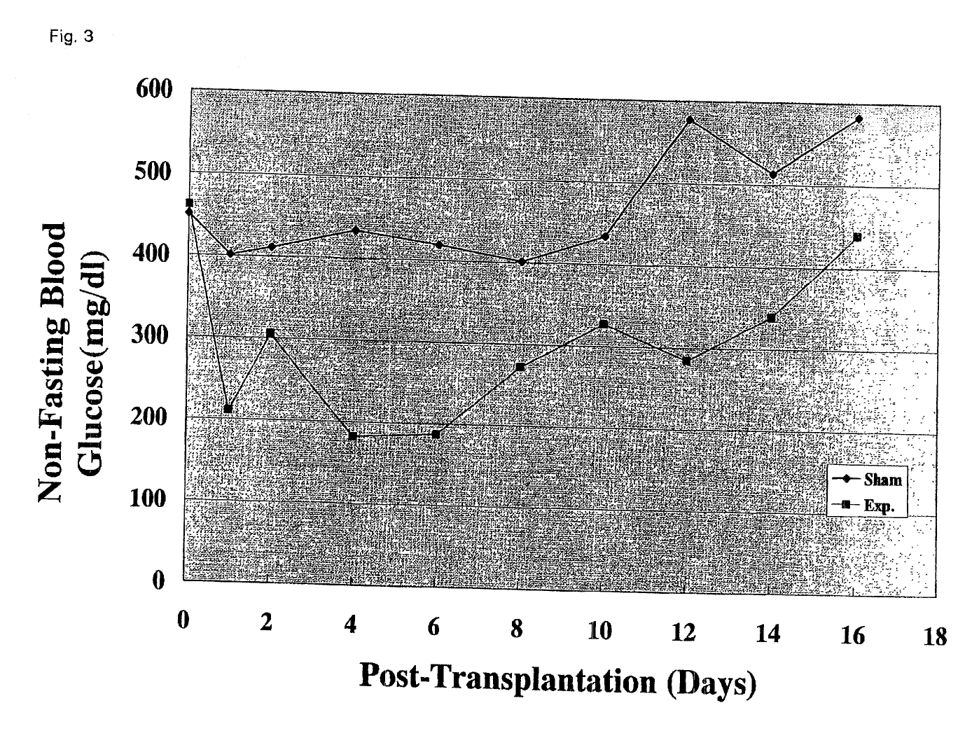

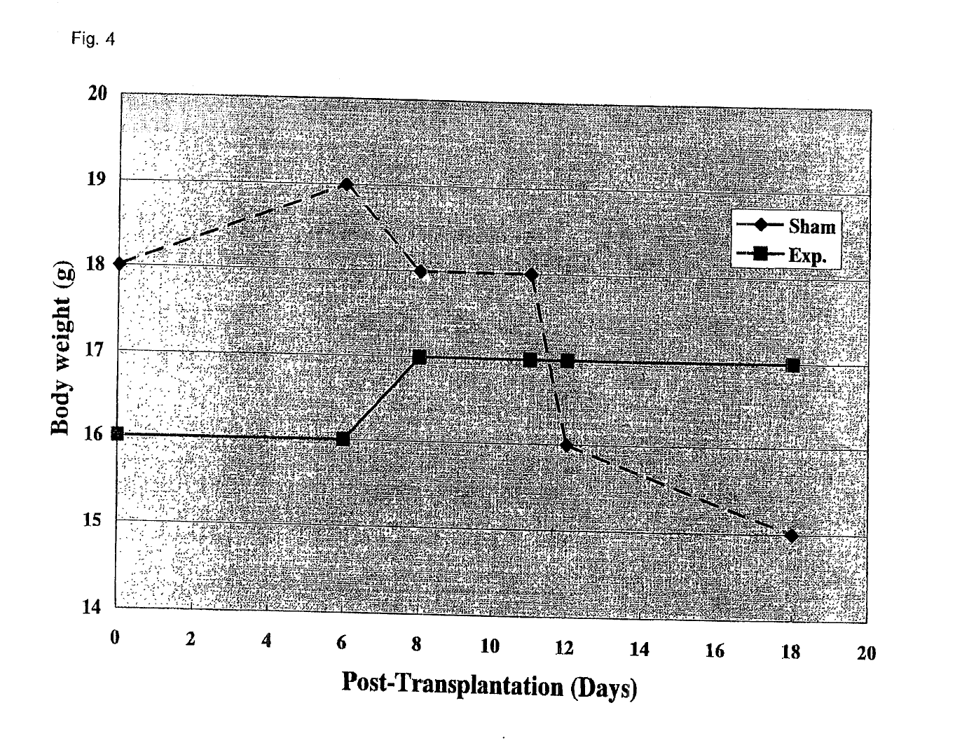

14 days after the STZ injection, the diabetic animals were grafted with 3000 insulin producing pancreatic islet like cell clusters obtained in Example 1 or received sham operation. Under nembutal anesthetization, the cell clusters suspended in PBS(−) were injected into the kidney subcapsuler region (one kidney) of the diabetic mice with 23-gauge winged needle. For the sham-operating group, the same volume of PBS(−) was injected in the same manner as above. The experimental group received cell clusters, non-treated control group and sham group consisted of 6, 3 and 3 animals respectively. After the transplantation, non-fasting blood glucose and body weight were monitored daily. The results are shown in FIGS. 3 and 4.

One day after the transplantation, the blood glucose of the experimental group significantly decreased and the significantly lower blood glucose level than the sham group was kept throughout the time of the experiment. The body weight of the implanted group increased slightly and kept stable.

At days 14 and 21, 2 and 4 animals implanted with the clusters were sacrificed respectively. All implanted mice remained healthy until killed and kept significantly lower blood glucose than the sham group. To the contrarily, blood glucose levels in non-treated control and sham groups were increased gradually and became exhausted. All of the mice of control and sham groups died prematurely from complication of diabetics between day 14 and day 30 of the operation.

From the sacrificed animals, the implanted tissue was excised, fixed with 4% paraformaldehyde in PBS and embedded in paraffin block. Thus obtained tissue slices of 4-8 μm thickness were immunohistochemically examined in the same manner as Example 1.

At the implanted region, single massed endocrine cells which were immunohistochemically positive to insulin and glucagone were observed. There was no teratoma observed at the area.

### EXAMPLE 3

Induction of Nerve Like Cells

The mouse ES cells same as used in Example 1 were treated in the same manner as steps 1-3 of Example 1. Thus obtained cell clusters were then cultured in a dish coated with poly-L-lysine and filled with serum free DMEM/F-12(1:1) medium (cat# 11320, Life Tech.) supplemented with 500 μg/ml of Bovine Insulin, 1 μg/ml of Progestron, 1600 μg/ml of Putrescine, 10 mM of lysine and 1 μg/ml of Laminin. The cells were cultured for 12 days and the obtained cells were examined genetically and immunohistochemically according to the same manner as described in Example 1. In this example, thyrosine hydroxylase(TH) polyclonal 1:200 (Pel-Freeze, Rogers, Ark.), thyrosine hydroxylase(TH) monoclonal 1:1000 (Sigma, St. Louis, Mo.), serotonin polyclonal 1:4000 (Sigma, St. Louis, Mo.), MAP 2 polyclonal (Chemicon International, Temecula, Calif.), and GFAP monoclonal (Clon Tech, Palo Alto, Calif.) were used in addition to the antibodies used in Example 1.

The obtained cells represented nerve fiber like appearance and were immunohistochemically positive to nestin, TuJ1 (β-tublin III), serotonin, GFAP, MAP 2 and tyrosine-hydroxylase.

By means of RT-PCR described in Example 1, the nerve like cells was confirmed to express Nurt-1 transcription factor. The primer sequences used herein for detecting Nurt-1 were as follows:

TGAAGAGAGC GGAGAAGGAG ATC
TCTGGAGTTA AGAAATCGGA GCTG
255 bp.

Accordingly, the obtained nerve like cells are capable of generating mature neurons if they are implanted in vivo.

### EXAMPLE 4

Human ES Cells

According to the same manner as described in Example 1, insulin producing pancreatic islet like cell clusters are obtained from human ES cells. ES cells may be those described in the art such as U.S. Pat. Nos. 5,843,780 and 6,200,806; Science 282, 1145-1147 (1998), the disclosures of the publications are herein incorporated by reference. Thus obtained cell clusters produce insulin and secret insulin in response to glucose in a dose dependent manner. The cell clusters are implanted into a human type I diabetic patient. About 3-5 10⁵ clusters are suspended in about 50-100 ml of Krebs-Ringer solution and the suspension is injected into the liver via portal vein, or is implanted to subcutaneous space as a bio-artificial pancreas. The pancreatic function of the implanted patient restores and the patient acquires insulin-independency.

What is claimed is:

1. A method for inducing differentiation of mammalian embryonic stem cells into functioning cells, which comprises the steps of;
1) culturing the mammalian embryonic stem cells together with feeder cells with a medium comprising leukemia inhibitor factor;
2) culturing the obtained cells in absence of feeder cells with a medium comprising leukemia Inhibitor factor and basic FGF in a suspension culture condition to give embryonic bodies;
3) culturing the obtained embryonic bodies with a selection-expanding medium; and
4) culturing the obtained cell clusters with a differentiation medium to give functioning cells.

2. The method of claim 1, wherein the medium used in step 2) comprises about 100-10000 U/ml of leukemia inhibitor factor.

3. The method of claim 1, wherein the medium used in step 2) comprises about 2-100 ng/ml of bFGF.

4. The method of claim 1, wherein the medium used in step 3) comprises nicotinamide, insulin and fibronectine in an serum-free cell culture medium.

5. The method of claim 1, wherein the functioning cells are insulin producing pancreatic islet like cell clusters.

6. The method of claim 5 wherein the medium used in step 4) comprises nicotinamide, insulin and laminine in a serum-free cell culture medium.

7. The method of claim 1, wherein the functioning cells are nerve like cells.

8. The method of claim 7 wherein the medium used in step 4) comprises L-lysine, insulin and laminine in a serum-free cell culture medium.

9. Functioning cells induced from mammalian ES cells by the method of claim 1.

10. Insulin secreting cell clusters induced from mammalian ES cells by the method of claim 5.

11. Nerve like cells induced from mammalian ES cells by the method of claim 7.

12. A method for treating a mammalian patient having disorders in pancreatic islet function, which comprises implanting pancreatic islet-like cell clusters induced from allogenic ES cells by the method of claim 5 to the patient.

13. The method of claim 12, wherein the patient is type I diabetic patient.

14. A method for treating a mammalian patient having disorders in nerve function, which comprises implanting nerve like cells induced from allogenic ES cells by the method of claim 7 to the patient.
